(19) United States
(12) Patent Application Publication
(10) Pub. No.: US 2004/0101492 A1
(43) Pub. Date: May 27, 2004

(54) Multicolored striped dentifrice composition

(75) Inventors: Lawrence Edward Dolan, Cincinnati, OH (US); Cloyd Dixon JR., Covington, KY (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: THE PROCTER & GAMBLE COMPANY

(21) Appl. No.: 10/626,947

(22) Filed: Jul. 25, 2003

Related U.S. Application Data
(60) Provisional application No. 60/429,075, filed on Nov. 26, 2002.

(51) Int. Cl. A61K 7/16

(52) U.S. Cl. 424/49

(57) ABSTRACT

The present invention relates to a multicolored, striped, dentifrice composition comprising:

a. a plurality of stripe phases comprising:

1. at least one first phase that is a translucent or transparent gel phase comprising an effective amount of colorant particles to create a striped appearance in the translucent or transparent gel phase; and

2. at least one second phase;

wherein the first phase and the second phase have contrasting colors and

wherein all of the phases are Theologically equivalent; and

b. a dentifrice dispensing means wherein the phases are in interfacial surface contact with the adjacent phase prior to extrusion from the dispensing means.

This application claims the benefit of U.S. Provisional application No. 60/429,075, filed on Nov. 26, 2002.

### TECHNICAL FIELD

This invention relates to an aesthetically pleasing and pleasant tasting multicolored dentifrice composition having two or more different stripe phases. More specifically this invention relates to a low cost and versatile striped toothpaste or gel wherein the two or more phases are not physically separated in the dispensing means, e.g. dentifrice tube. Furthermore, at least one stripe phase is created via the incorporation of a low concentration of entrained or encapsulated colored or white particles into a transparent or translucent gel matrix.

### BACKGROUND ART

Aesthetic effects are known to play an important role in consumer acceptance of dentifrice products. These aesthetic effects (color, flavor, etc.) allow consumers to distinguish particular products in the marketplace and to identify products having desirable, pleasing and unique properties versus other available consumer products. Aesthetics also promote the consumer's recognition and use of the product. Contrasting colored stripes or speckles are known in dentifrice products. Such stripes or speckles provide an aesthetic effect, which the user finds pleasing and promotes the use of the dentifrice. For example, striped dentifrice products containing water-soluble dyes are known in the prior art, such as those disclosed in U.S. Pat. Nos. 4,358,437, 4,568,534, and 4,487,757. The use of these water-soluble dyes, however, results invisible bleeding of the dye color between the phases.

The problem of bleeding or migration of color from one stripe into another stripe component is especially severe if one colored component is applied to the surface of a white base. For this reason, a colorant that exhibits substantially no visible bleeding is required.

The prior art suggests several ways to reduce color migration and bleeding in striped dentifrice compositions. For example, U.S. Pat. No. 5,876,701, Wong et al, issued Mar. 2, 1999, teaches a striped and/or speckled dentifrice stable to color bleeding, wherein at least one dentifrice component contains a colorant entrained in a high density polyethylene matrix having a melting point range as measured by DSC is between about 110° C. and about 145° C. Also, U.S. Pat. Nos. 3,957,964, 3,929,988, 4,071,614 and 4,348,378 disclose dentifrices containing encapsulated ingredients such as flavors. These ingredients are maintained separate from other dentifrice ingredients during manufacture and storage. The encapsulated ingredients are then released into the dentifrice during brushing by the consumer. Furthermore, the prior art also teaches to encapsulate water insoluble dyes in capsules wherein the shell material is formed from non-toxic naturally occurring waxes such as carnauba wax, candelella wax, castor wax, paraffin wax and bayberry wax in U.S. Pat. No. 4,202,878.

Accordingly, there is a need for an alternative composition and process for producing striped and speckled dentifrices, which will essentially reduce processing costs while still providing substantially no visible colorant bleeding. This invention relates to an aesthetically pleasing and pleasant tasting striped and speckled toothpaste or gel wherein there is substantially no colorant bleeding between dentifrice phases. This multicolored dentifrice composition comprises striped phases that are not physically separated in the dispensing means, e.g. dentifrice tube. Furthermore, at least one stripe phase is created via the incorporation of a low concentration of entrained or encapsulated particles into a transparent or translucent gel matrix.

### SUMMARY OF THE INVENTION

The present invention relates to a multicolored, striped, dentifrice composition comprising:

a. a plurality of stripe phases comprising:

1. at least one first phase that is a translucent or transparent gel phase comprising an effective amount of colorant particles to create a striped appearance in the translucent or transparent gel phase; and

2. at least one second phase;

wherein the first phase and the second phase have contrasting colors and

wherein all of the phases are Theologically equivalent; and

b. a dentifrice dispensing means wherein the phases are in interfacial surface contact with the adjacent phase prior to extrusion from the dispensing means.

### DETAILED DESCRIPTION

Definitions

By “anticalculus” or “antitartar” agent, as used herein, means a material effective in reducing, controlling, inhibiting, preventing, and/or minimizing mineral (e.g., calcium phosphate) deposition related to calculus or tartar formation.

By “color” as used herein, means any visually perceivable color, such as white, red, blue, yellow, green, etc.

A “plurality of stripe phases” as used herein means that the composition has 2 or more stripes.

By “multicolored” or “contrasting colors” as used herein means that each different phase has a different color, that the phases have at least 2 different colors, or that the phases have at least 2 different shades or hues of the same color. Where the phases have at least 2 different shades or hues of the same color, these different shades or hues must give a striped appearance upon visual observation.

By “safe and effective amount” as used herein is meant an amount of a component, high enough to significantly (positively) modify the condition to be treated, but low enough to avoid serious side effects (at a reasonable benefit/risk ratio), within the scope of sound medical/dental judgment. The safe and effective amount of a component, will vary with the particular condition being treated, the age and physical condition of the patient being treated, the severity of the condition, the duration of treatment, the nature of concurrent therapy, the specific form employed, and the particular vehicle from which the component is applied.

By “oral care composition” or “oral composition” as used herein is meant a product which is not intentionally swallowed for purposes of systemic administration of therapeutic agents, but is retained in the oral cavity for a sufficient time to contact substantially all of the dental surfaces and/or oral mucosal tissues for purposes of oral activity.

All percentages and ratios used hereinafter are by weight of total composition, unless otherwise indicated.

All measurements referred to herein are made at 25° C. unless otherwise specified. All percentages, ratios, and levels of ingredients referred to herein are based on the actual amount of the ingredient, and do not include solvents, fillers, or other materials with which the ingredient may be combined as a commercially available product, unless otherwise indicated.

All publications, patent applications, and issued patents mentioned herein are hereby incorporated in their entirety by reference. Citation of any reference is not an admission regarding any determination as to its availability as prior art to the claimed invention.

Herein, “comprising” means the term “comprising” and can include “consisting of” and “consisting essentially of.”

### Colorant Particles

The color present in the translucent or transparent gel phase is provided only by the colorant particles such that there is no other colorant or dye contained in the translucent or transparent gel phase. In one embodiment the colorant particles are color entrained or encapsulated color particles. A variety of materials can be used to entrain or encapsulate the color particles. These include high density polyethylene (HDPE), low density polyethylene (LDPE), natural waxes, gelling agents, synthetic waxes, water soluble thermoplastic polymers such as non-emulsifiable grade polyethylene, insoluble binders such as ethyl cellulose, water soluble binders such as polyvinylpyrrolidone, and mixtures thereof.

The level of colorant particle in the composition of the present invention is from about 0.05% to about 5%, in another embodiment from about 0.1% to about 3%, and in another embodiment from about 0.15% to about 2% and in yet another embodiment from about 0.5% to about 1.5%, by weight of the composition. In one embodiment, in order to sufficiently color the transparent or translucent gel phase, this gel phase contains from about 0.5% to about 2%, in another embodiment from 0.6 to about 1.5%, and in another embodiment from about 0.7% to about 1% by weight of colorant particles.

Specific color entrained or encapsulated color particles and methods of making them are disclosed in the following references. HDPE is disclosed in U.S. Pat. No. 5,876,701, Wong et al, issued Mar. 2, 1999, which teaches that at least one dentifrice component contains a colorant entrained in a high density polyethylene matrix having a melting point range as measured by DSC between about 110° C. and about 145° C. Colored entrained synthetic waxes are disclosed in U.S. Pat. No. 6,315,986, Wong et al., issued Nov. 13, 2001.

Insoluble binders such as ethyl cellulose used alone or in combination with water soluble binders such as polyvinylpyrrolidone, agglomerated with water insoluble powdered functional materials including colorants are disclosed in U.S. Pat. Nos. 4,443,564, Hauschild et al., issued Apr. 17, 1984, U.S. Pat. No. 4,444,570, Barth et al., issued Apr. 24, 1984, and U.S. Pat. No. 4,663,152, Barth et al., issued May 5, 1987.

Agglomerates of natural waxes and gelling agents are disclosed in U.S. Pat. No. 4,202,878, Ritze, issued May 13, 1980. Suitable waxes disclosed in U.S. Pat. No. 4,202,878 include those which are non-toxic and have a hardness value of from about 0 to about 65 (ASTM Test D 1321-65) and preferably from about 0 to 5. Examples of suitable waxes include carnauba wax, candelilla wax, purified montan wax, castor wax, paraffin, ceresin wax and bayberry wax. Preferred waxes have a melting point above 70° F., more preferably from 180° to 230° F. Additional waxes having these properties which can be used herein are disclosed in Soap & Chem. Specialties, Vol. 33, page 141 (1957). See also Industrial Waxes, Vol. I and II, H. Bennett, Chemical Publishing Co., Inc., New York, 1963, for a discussion of waxes and their properties. Also disclosed in US '878 are suitable gelling agents that form stable, firm gels which are: non-toxic; hard enough to withstand conventional shear stresses when admixed in the dentifrice or extruded from the toothpaste tube; frangible enough to disintegrate into smaller particles at time of use to give no adverse mouth impression; light colored; innocuous in flavor and odor; and compatible with toothpaste ingredients. Gelling agents which can be used to form agglomerates of pigment particles include agar-agar, cassaya starch, Avicel (microcrystalline cellulose by FMC Corp.), and high molecular weight carboxyvinyl polymers such as Carbopol 940, supplied by the B. F. Goodrich Chemical Company.

In addition U.S. Pat. Nos. 4,007,259 and U.S. Pat. No. 3,928,559, teach particles having a water-soluble thermoplastic polymer such as non-emulsifiable grade of polyethylene for the speckling of dentifrice.

In addition speckles having insoluble binders taught in U.S. Pat. No. 4,069,311, Mannara, issued Jan. 17, 1978, can also be used herein.

The color-entrained particles as used herein have, in one embodiment, a mean particle size within the range from about 200 to about 500 microns, in another embodiment from about 250 microns to about 350 microns, in yet another embodiment from about 270 to about 320 microns.

Colorants used in the present colorant particles are selected from the group consisting of pigments, dyes, lakes, and mixtures thereof. The dyes used in the practice of the present invention are distributed uniformly throughout the dentifrice phase(s).

Pigments used in the practice of the present invention include non-toxic, water insoluble inorganic pigments such as titanium dioxide, chromium oxide greens, ultramarine blues and pinks, ferric oxides, Eye shadow Blue KO, Colour Index 77 510, EG-No., Blue 15 (C-Blue 17), dyed cellulose particles such as dyed cotton linters and dyed wood pulp.

The dyes used in the present colorant particles include natural or synthetic dyes of the types permitted in foods and drugs, such as those listed in Title 21 of the U.S. Code of Federal Regulations, Section 74, and are generally food color additives presently certified under the Food Drug & Cosmetic Act for use in food and ingested drugs, including dyes such as FD&C Red No. 3 (sodium salt of tetraiodofluorescein), FD&C Yellow No. 5 (sodium salt of 4-p-sulfophenylazo-1-p-sulfophenyl-5-hydroxypyrazole-3 carboxylic acid), FD&C Yellow No. 6 (sodium salt of p-sulfophenylazo-B-naphtol-6-monosulfonate), FD&C Green No. 1, FD&C Green No. 3 (disodium salt of 4-{[4-(N-ethyl-p-sulfobenzylaino)-phenyl]-(4-hydroxy-2-sulfoniumphenyl)-methylene}-[1-(N-ethyl-N-p-sulfobenzyl)-Δ-3,5cyclohexadienimine], FD&C Blue No. 1 (disodium salt of dibenzyldiethyldiaminotriphenylcarbinol trisulfonic acid anhydrite), FD&C Blue No. 2(sodium salt of disulfonic acid of indigotin), FD&C Yellow No. 10, and mixtures thereof, and lakes thereof. Mixtures of water insoluble dyes with water-soluble dyes can also be used in the present invention.

Preferred colorants comprise about 0.1% to about 40% by weight, preferably about 7% to about 30% by weight, of a water soluble dye on a substrate such as alumina, zirconia and titania and preferably alumina hydrate.

The first phase and the second phase can comprise the same or different oral care active agents or topical oral care carriers as described herein. The color present in the translucent or transparent gel phase is provided only by the colorant particles such that there is no other colorant or dye contained in the translucent or transparent gel phase. The second phase can also be a gel wherein the color is provided only by colorant particles such that there is no other colorant or dye contained in the second phase (as long as it has a contrasting color). The second phase can also contain TiO₂ (or other whitening pigment) such that the second phase is a white stripe. The second phase can also contain dyes, pigments, e.g. colorants of any particle size that are not encapsulated or entrained colorants.

In one embodiment the second phase comprises TiO₂ (or other whitening pigment) wherein the second phase also comprises the same colorant particle of the same or similar particle size as the first phase, wherein the first phase comprises from about 0.6 to about 1% colorant particle and the second phase comprises from about 0.2 to about 0.5% by weight of colorant particles.

### Rheology of Phases

The different phases of the composition have similar rheological properties. In one embodiment the different phases of the composition are rheologically equivalent. The term “Theologically equivalent” as used herein means that the viscosity of the different phases should vary no more than about 20%, in another embodiment no more than about 10%, in yet another embodiment no more than about 5% from the viscosity of any other phase. The theological equivalency of the different phases of the present dentifrice composition assists in preventing the mixing of ingredients across the interfacial boundary of the phase layers of the different phases.

The viscosity of the phases of the present composition is from about 18 Brookfield units to about 60 Brookfield units (torque %), at about 25 degrees C. using a Brookfield Viscometer (Model RV111), spindle E, at a rotation of 2.5 r.p.m.

The viscosity is such that, after extrusion from the dispensing means, the composition substantially retains its original viscosity over a period of about 1 to about 5 minutes.

### Method of Making Composition and Dispensing Means

In accordance with the present invention there is provided a striped, multicolored dentifrice composition comprising at least two stripe phases. The different phases are arranged in opposed, side-by-side, parallel layers in interfacial contact with the adjacent phases prior to extrusion, when placed in the dispensing means. During storage the different phases of the present composition demonstrate little or no tendency for any of the ingredients (e.g. colors) to bleed or diffuse from one phase to another. Therefore, the use of conventional dentifrice tube and packaging means are possible thus reducing packaging and manufacturing costs.

Several techniques for striping dentifrice compositions are known in the art. The method for striping the composition herein can be selected from any of the known methods, as long as the method does not physically separate the different phases in the dispensing means. For example, dentifrice striping can be accomplished by deep striping. In deep striping methods, the layers of striping and base material are in interfacial contact with each other or are juxtaposition in the dispenser in the pattern of the desired stripes, hence the initial deposition of the stripe phase on a base dentifrice phase occurs prior to extrusion from the dispenser.

In one embodiment the quantity of the gel phase (first phase) to the second phase is generally in the ratio of about 20:80 to about 80:20 and in another embodiment the range is from about 40:60 to about 60:40 and in yet another embodiment is about 50:50.

The dispensing means can be any collapsible tube or other containers (e.g. pumps or bottles) known in the art (e.g. aluminum collapsible tubes with capped openings) for dispensing dentifrice compositions, as long as the dispensing means does not physically separate gel phases from the other phases. The collapsible tube is charged with the different phases of the composition by apparatus and methods of filling as described for example in British Patent Specification No. 962,757. In one embodiment a white paste phase (second phase) is centrally located and the gel phase is dispensed from orientation spaced circumferentially around the central tube. The translucent or transparent gel may be dispensed from two tubes located diametrically opposite to the central tube. A toothpaste with a white paste phase and alternating color gel stripes can then be obtained.

In one embodiment the different phases contain essentially identical ingredients except the second phase contains TiO2 and the gel first phase is free of TiO2 but has colorant particles. A dentifrice with a first phase and a second phase can produce a dentifrice with 2,4,6,8, etc. strips. Where 3 phases are used, one can obtain a dentifrice with 3,6,9, etc. stripes.

In one embodiment the dispensing means is a deformable tube of clear or translucent synthetic organic polymeric material such as polyvinyl chloride, polyethylene, polyvinylidine chloride or similar material. A clear dispensing means allows the consumer to see the speckled and striped appearance of the dentifrice.

### Optional Oral Care Active Agents

The present invention may optionally comprise a safe and effective amount of an oral care active agent selected from the group consisting of anticalculus agent, fluoride ion source, antimicrobial agents, dentinal desensitizing agents, anesthetic agents, antifungal agents, anti-inflammatory agents, selective H-2 antagonists, anticaries agents, remineralization agents, and mixtures thereof.

The oral care active agents can be present in the compositions in safe and effective amounts. These amounts will be known by those skilled in the art and are disclosed below. The oral care active agents can be present in one or both the first phase and/or the second phase of the present compositions.

Anticaries Agents and Fluoride Ion Source

The active agent may include a fluoride ion source, providing free fluoride ions during the use of the composition. In one embodiment the fluoride ion source is selected from the group consisting of sodium fluoride, stannous fluoride, indium fluoride, organic fluorides such as amine fluorides, and sodium monofluorophosphate. Sodium fluoride is the fluoride ion in another embodiment. Norris et al., U.S. Pat. No. 2,946,725, issued Jul. 26, 1960, and Widder et al., U.S. Pat. No. 3,678,154 issued Jul. 18, 1972, disclose such salts as well as others that can be used as the fluoride ion source. These patents are incorporated herein by reference in their entirety.

The present composition may optionally comprise a safe and effective amount of a fluoride ion source. In another embodiment the level is from about 50 ppm to about 3500 ppm, in another embodiment from about 100 ppm to about 3000 ppm, and in another embodiment from about 200 ppm to about 2,800 ppm, and in another embodiment from about 500 ppm to about 1,500 ppm, of free fluoride ions.

Anticalculus Agents

The present compositions may optionally comprise a safe and effective amount of at least one anticalculus agent. This amount is generally from about 0.01% to about 40% by weight of the composition, in another embodiment is from about 0.1% to about 25%, and in yet another embodiment is from about 4.5% to about 20%, and in yet another embodiment is from about 5% to about 15%, by weight of the composition. The anticalculus agent should also be essentially compatible with the other components of the composition.

The anticalculus agent is selected from the group consisting of polyphosphates and salts thereof; polyamino propane sulfonic acid (AMPS) and salts thereof; polyolefin sulfonates and salts thereof; polyvinyl phosphates and salts thereof; polyolefin phosphates and salts thereof; diphosphonates and salts thereof; phosphonoalkane carboxylic acid and salts thereof; polyphosphonates and salts thereof; polyvinyl phosphonates and salts thereof; polyolefin phosphonates and salts thereof; polypeptides; and mixtures thereof. In one embodiment, the salts are alkali metal salts. In another embodiment the anticalculus agent is selected from the group consisting of polyphosphates and salts thereof; diphosphonates and salts thereof; and mixtures thereof. In another embodiment the anticalculus agent is selected from the group consisting of pyrophosphate, polyphosphate, and mixtures thereof.

Polyphosphate

In one embodiment of the present invention, the anticalculus agent is a polyphosphate. A polyphosphate is generally understood to consist of two or more phosphate molecules arranged primarily in a linear configuration, although some cyclic derivatives may be present. Linear polyphosphates correspond to (X PO₃) n where n is about 2 to about 125; wherein preferably n is greater than 4, and X is for example sodium, potassium, etc. For (X PO₃)ₙ when n is at least 3 the polyphosphates are glassy in character. Counterions for these phosphates may be the alkali metal, alkaline earth metal, ammonium, C₂-C₆ alkanolammonium and salt mixtures. Polyphosphates are generally employed as their wholly or partially neutralized water-soluble alkali metal salts such as potassium, sodium, ammonium salts, and mixtures thereof. The inorganic polyphosphate salts include alkali metal (e.g. sodium) tripolyphosphate, tetrapolyphosphate, dialkyl metal (e.g. disodium) diacid, trialkyl metal (e.g. trisodium) monoacid, potassium hydrogen phosphate, sodium hydrogen phosphate, and alkali metal (e.g. sodium) hexametaphosphate, and mixtures thereof. Polyphosphates larger than tetrapolyphosphate usually occur as amorphous glassy materials. In one embodiment the polyphosphates are those manufactured by FMC Corporation which are commercially known as Sodaphos (n=6), Hexaphos (n=13), and Glass H (n=21), and mixtures thereof. The polyphosphate source will typically comprise from about 0.5% to about 20%, in one embodiment from about 4% to about 15%, in yet another embodiment from about 6% to about 12%, by weight of the composition.

The phosphate sources are described in more detail in Kirk & Othmer, Encyclopedia of Chemical Technology, Fourth Edition, Volume 18, Wiley-Interscience Publishers (1996), pages 685-707, incorporated herein by reference in its entirety, including all references incorporated into Kirk & Othmer.

In one embodiment the polyphosphates are the linear “glassy” polyphosphates having the formula:

XO(XPO₃)ₙX

wherein X is sodium or potassium; and n averages from about 6 to about 125.

In one embodiment, when n is at least 2 in either of the above polyphosphate formulas, the level of anticalculus agent is from about 4.5% to about 40%, in another embodiment is from about 5% to about 25%, and in even another embodiment is from about 8% to about 15%, by weight of the composition. Polyphosphates are disclosed in U.S. Pat. No. 4,913,895, herein incorporated by reference.

Pyrophosphate

The pyrophosphate salts useful in the present compositions include, alkali metal pyrophosphates, di-, tri-, and mono-potassium or sodium pyrophosphates, dialkali metal pyrophosphate salts, tetraalkali metal pyrophosphate salts, and mixtures thereof. In one embodiment the pyrophosphate salt is selected from the group consisting of trisodium pyrophosphate, disodium dihydrogen pyrophosphate (Na₂H₂P₂O₇), dipotassium pyrophosphate, tetrasodium pyrophosphate (Na₄P₂O₇), tetrapotassium pyrophosphate (K₄P₂O₇), and mixtures thereof. The pyrophosphate salts described in U.S. Pat. No. 4,515,772, issued May 7, 1985, and U.S. Pat. No. 4,885,155, issued Dec. 5, 1989, both to Parran et al., are incorporated herein by reference in their entirety, as well as the references disclosed therein. The pyrophosphate salts are described in more detail in Kirk & Othmer, Encyclopedia of Chemical Technology, Third Edition, Volume 17, Wiley-Interscience Publishers (1982), pages 685-707, incorporated herein by reference in its entirety, including all references incorporated into Kirk & Othmer.

In one embodiment, the compositions of the present invention comprise tetrasodium pyrophosphate. Tetrasodium pyrophosphate may be the anhydrous salt form or the decahydrate form, or any other species stable in solid form in the present compositions. The salt is in its solid particle form, which may be its crystalline and/or amorphous state, with the particle size of the salt preferably being small enough to be aesthetically acceptable and readily soluble during use.

The level of pyrophosphate salt in the compositions of the present invention is any safe and effective amount, and is generally from about 1.5% to about 15%, in another embodiment from about 2% to about 10%, and yet in another embodiment from about 3% to about 8%, by weight of the composition.

Other Anticalculus Agents

Polyolefin sulfonates include those wherein the olefin group contains 2 or more carbon atoms, and salts thereof. Polyolefin phosphonates include those wherein the olefin group contains 2 or more carbon atoms. Polyvinylphosphonates include polyvinylphosphonic acid. Diphosphonates and salts thereof include azocycloalkane-2,2-diphosphonic acids and salts thereof, ions of azocycloalkane-2,2-diphosphonic acids and salts thereof (such as those which the alkane moiety has five, six or seven carbon atoms, in which the nitrogen atom is unsubstituted or carries a lower alkyl substitutent, e.g. methyl), azacyclohexane-2,2-diphosphonic acid, azacyclopentane-2,2-diphosphonic acid, N-methyl-azacyclopentane-2,3-diphosphonic acid, EHDP (ethanehydroxy-1,1, -diphosphonic acid), AHP (azacycloheptane-2,2-diphosphonic acid, a.k.a. 1-azocycloheptylidene-2,2-diphosphonic acid), ethane-1-amino-1,1-diphosphonate, dichloromethane-diphosphonate, etc. Phosphonoalkane carboxylic acid or their alkali metal salts include PPTA (phosphonopropane tricarboxylic acid), PBTA (phosphonobutane-1,2,4-tricarboxylic acid), each as acid or alkali metal salts. Polyolefin phosphates include those wherein the olefin group contains 2 or more carbon atoms. Polypeptides include polyaspartic and polyglutamic acids.

Azacycloalkane-2,2-diphosphonic acids are disclosed in U.S. Pat. No. 3,941,772, issued Mar. 2, 1976, Ploger et al., assigned to Henkel and U.S. Pat. No. 3,988,443, issued Oct. 26, 1976, Ploger et al., which are herein incorporated by reference in their entirety.

Optional agents to be used in place of or in combination with the pyrophosphate salt include such known materials as synthetic anionic polymers, including polyacrylates and copolymers of maleic anhydride or acid and methyl vinyl ether (e.g., Gantrez), as described, for example, in U.S. Pat. No. 4,627,977, to Gaffar et al., the disclosure of which is incorporated herein by reference in its entirety; as well as, e.g., polyamino propoane sulfonic acid (AMPS), zinc citrate trihydrate, polyphosphates (e.g., tripolyphosphate; hexametaphosphate), diphosphonates (e.g., EHDP; AHP), polypeptides (such as polyaspartic and polyglutamic acids), and mixtures thereof.

Antimicrobial Agents, Desensitizing Agents, Anesthetic Agents, Antifungal Agents

Antimicrobial antiplaque agents may also be optionally present in the present compositions. Such agents may include, but are not limited to, triclosan, 5-chloro-2-(2,4-dichlorophenoxy)-phenol, as described in The Merck Index, 11th ed. (1989), pp. 1529 (entry no. 9573) in U.S. Pat. No. 3,506,720, and in European Patent Application No. 0,251,591 of Beecham Group, PLC, published Jan. 7, 1988; chlorhexidine (Merck Index, no. 2090), alexidine (Merck Index, no. 222; hexetidine (Merck Index, no. 4624); sanguinarine (Merck Index, no. 8320); benzalkonium chloride (Merck Index, no. 1066); salicylanilide (Merck Index, no. 8299); domiphen bromide (Merck Index, no. 3411); cetylpyridinium chloride (CPC) (Merck Index, no. 2024; tetradecylpyridinium chloride (TPC); N-tetradecyl-4-ethylpyridinium chloride (TDEPC); octenidine; delmopinol, octapinol, and other piperidino derivatives; effective antimicrobial amounts of essential oils and combinations thereof for example citral, geranial, and combinations of menthol, eucalyptol, thymol and methyl salicylate; antimicrobial metals and salts thereof for example those providing zinc ions, stannous ions, copper ions, and/or mixtures thereof; bisbiguanides, or phenolics; antibiotics such as augmentin, amoxicillin, tetracycline, doxycycline, minocycline, and metronidazole; and analogs and salts of the above antimicrobial antiplaque agents. Dentinal desensitizing agents include agents such as potassium nitrate, strontium chloride. Anesthetic agents include agents such as lidocaine or benzocaine; antifungals such as those for the treatment of candida albicans. If present, these agents generally are present in a safe and effective amount, for example from about 0.1% to about 5% by weight of the compositions of the present invention.

Anti-Inflammatory Agents

Anti-inflammatory agents may also be present in the compositions of the present invention. Such agents may include, but are not limited to, non-steroidal anti-inflammatory agents such as aspirin, ketorolac, flurbiprofen, ibuprofen, naproxen, indomethacin, aspirin, ketoprofen, piroxicam and meclofenamic acid, COX-2 inhibitors such as valdecoxib, celecoxib and rofecoxib, and mixtures thereof. If present, the anti-inflammatory agents generally comprise from about 0.001% to about 5% by weight of the compositions of the present invention. Ketorolac is described in U.S. Pat. No. 5,626,838, issued May 6, 1997, incorporated herein by reference in its entirety.

H-2 Antagonists

The present invention may also comprise a safe and effective amount of a selective H-2 antagonist including compounds disclosed in U.S. Pat. No. 5,294,433, Singer et al., issued Mar. 15, 1994, which is herein incorporated by reference in its entirety.

Topical, Oral Carrier

The present invention optionally comprises topical oral carriers in one or both the first phase and/or the second phase of the present compositions. The carriers of the present invention may include the usual and conventional components of toothpastes and tooth gels, as more fully described hereinafter.

By “pharmaceutically-acceptable topical oral carrier,” or “topical, oral carrier” as used herein, is meant one or more compatible solid or liquid filler diluents or encapsulating substances which are suitable for topical, oral administration. By “compatible,” as used herein, is meant that the components of the composition are capable of being commingled without interaction in a manner which would substantially reduce the composition's stability and/or efficacy for treating or preventing oral care conditions, according to the compositions and methods of the present invention.

“Toothpaste carriers” are disclosed in, e.g., U.S. Pat. No. 3,988,433, to Benedict, the disclosure of which is incorporated herein by reference (e.g., abrasive materials, sudsing agents, binders, humectants, flavoring and sweetening agents, etc.). Carriers suitable for the preparation of compositions of the present invention are well known in the art. Their selection will depend on secondary considerations like taste, cost, and shelf stability, etc.

Compositions of the subject invention are in the form of dentifrices, such as toothpastes and tooth gels. Components of such toothpaste and tooth gels generally include one or more of a dental abrasive (from about 10% to about 50%), a surfactant (from about 0.5% to about 10%), a thickening agent (from about 0.1% to about 5%), a humectant (from about 10% to about 55%), a flavoring agent (from about 0.04% to about 2%), a sweetening agent (from about 0.1% to about 3%), a coloring agent (from about 0.01% to about 0.5%) and water (from about 2% to about 45%). Such toothpaste or tooth gel may also include one or more of an additional anticaries agent (from about 0.05% to about 10% additional anticaries agent), and an anticalculus agent (from about 0.1% to about 13%).

Types of carriers which may be included in compositions of the present invention, along with specific non-limiting examples, are:

Abrasives

Dental abrasives useful in the topical, oral carriers of the compositions of the subject invention include many different materials. The material selected must be one which is compatible within the composition of interest and does not excessively abrade dentin. Suitable abrasives include, for example, silicas including gels and precipitates, insoluble sodium polymetaphosphate, hydrated alumina, calcium carbonate, dicalcium orthophosphate dihydrate, calcium pyrophosphate, tricalcium phosphate, calcium polymetaphosphate, and resinous abrasive materials such as particulate condensation products of urea and formaldehyde.

Another class of abrasives for use in the present compositions is the particulate thermosetting polymerized resins as described in U.S. Pat. No. 3,070,510 issued to Cooley & Grabenstetter on Dec. 25, 1962. Suitable resins include, for example, melamines, phenolics, ureas, melamine-ureas, melamine-formaldehydes, urea-formaldehyde, melamine-ureaformaldehydes, cross-linked epoxides, and cross-linked polyesters.

Silica dental abrasives of various types are preferred because of their unique benefits of exceptional dental cleaning and polishing performance without unduly abrading tooth enamel or dentine. The silica abrasive polishing materials herein, as well as other abrasives, generally have an average particle size ranging between about 0.1 to about 30 microns, and preferably from about 5 to about 15 microns. The abrasive can be precipitated silica or silica gels such as the silica xerogels described in Pader et al., U.S. Pat. No. 3,538,230, issued Mar. 2, 1970, and DiGiulio, U.S. Pat. No. 3,862,307, issued Jan. 21, 1975, both incorporated herein by reference in their entirety. Preferred are the silica xerogels marketed under the trade name “Syloid” by the W.R. Grace & Company, Davison Chemical Division. Also preferred are the precipitated silica materials such as those marketed by the J. M. Huber Corporation under the trade name, Zeodent®, particularly the silica carrying the designation Zeodent 119®. The types of silica dental abrasives useful in the toothpastes of the present invention are described in more detail in Wason, U.S. Pat. No. 4,340,583, issued Jul. 29, 1982. The abrasive in the toothpaste compositions described herein is generally present at a level of from about 6% to about 70% by weight of the composition. Preferably, toothpastes contain from about 10% to about 50% of abrasive, by weight of the composition.

A particularly preferred precipitated silica is the silica disclosed in U.S. Pat. No. 5,603,920, issued on Feb. 18, 1997; U.S. Pat. No. 5,589,160, issued Dec. 31, 1996; U.S. Pat. No. 5,658,553, issued Aug. 19, 1997; U.S. Pat. No. 5,651,958, issued Jul. 29, 1997, all of which are assigned to the Procter & Gamble Co. All of these patents are incorporated herein by reference in their entirety. Mixtures of abrasives can be used.

Thickening Agents

In preparing toothpaste or gels, it may be generally necessary to add some thickening material to provide a desirable consistency of the composition, to provide desirable release characteristics upon use, to provide shelf stability, and to provide stability of the composition, etc. Preferred thickening agents are carboxyvinyl polymers, carrageenan, hydroxyethyl cellulose, laponite and water soluble salts of cellulose ethers such as sodium carboxymethylcellulose and sodium carboxymethyl hydroxyethyl cellulose. Natural gums such as gum karaya, xanthan gum, gum arabic, and gum tragacanth can also be used. Colloidal magnesium aluminum silicate or finely divided silica can be used as part of the thickening agent to further improve texture.

Thickening agents can include polymeric polyether compounds, e.g., polyethylene or polypropylene oxide (M.W. 300 to 1,000,000), capped with alkyl or acyl groups containing 1 to about 18 carbon atoms.

A preferred class of thickening or gelling agents includes a class of homopolymers of acrylic acid crosslinked with an alkyl ether of pentaerythritol or an alkyl ether of sucrose, or carbomers. Carbomers are commercially available from B.F. Goodrich as the Carbopol® series. Particularly preferred carbopols include Carbopol 934, 940, 941, 956, and mixtures thereof.

Thickening agents, in an amount from about 0.1% to about 15%, in another embodiment from about 0.2% to about 6%, in yet another embodiment from about 0.4% to about 5%, by weight of the total toothpaste composition, can be used.

Humectants

Another optional component of the topical, oral carriers of the compositions of the subject invention is a humectant. The humectant serves to keep toothpaste compositions from hardening upon exposure to air, to give compositions a moist feel to the mouth, and, for particular humectants, to impart desirable sweetness of flavor to toothpaste compositions. The humectant, on a pure humectant basis, generally comprises from about 0% to about 70%, preferably from about 5% to about 25%, by weight of the compositions herein. Suitable humectants for use in compositions of the subject invention include edible polyhydric alcohols such as glycerin, sorbitol, xylitol, butylene glycol, polyethylene glycol, and propylene glycol, especially sorbitol and glycerin.

Alkali Metal Bicarbonate Salt

The present invention may also include an alkali metal bicarbonate salt. Alkali metal bicarbonate salts are soluble in water and unless stabilized, tend to release carbon dioxide in an aqueous system. Sodium bicarbonate, also known as baking soda, is the preferred alkali metal bicarbonate salt. The present composition may contain from about 0.5% to about 30%, in one embodiment from about 0.5% to about 15%, and in another embodiment from about 0.5% to about 5% of an alkali metal bicarbonate salt.

Miscellaneous Carriers

Water employed in the preparation of commercially suitable oral compositions herein should preferably be of low ion content and free of organic impurities. Water generally comprises from about 5% to about 70%, in another embodiment from about 10 to about 50%, in another embodiment from about 11% to about 30%, and in yet another embodiment from about 15% to about 25%, by weight of the composition herein. These amounts of water include the free water which is added plus that which is introduced with other materials, such as with sorbitol. To aid in achieving clarity of the first phase, lower water levels are preferred.

Titanium dioxide may also be added to the present composition. Titanium dioxide is a white powder which adds opacity to the compositions. Titanium dioxide generally comprises from about 0.1 to 5%, in another embodiment from about 0.25% to about 1% by weight of the dentifrice compositions.

Other optional agents include synthetic anionic polymeric polycarboxylates being employed in the form of their free acids or partially or preferably fully neutralized water soluble alkali metal (e.g. potassium and preferably sodium) or ammonium salts and are disclosed in U.S. Pat. No. 4,152,420 to Gaffar, U.S. Pat. No. 3,956,480 to Dichter et al., U.S. Pat. No. 4,138,477 to Gaffar, U.S. Pat. No. 4,183,914 to Gaffar et al., and U.S. Pat. No. 4,906,456 to Gaffar et al., all of which are incorporated herein by reference in their entirety. Preferred are 1:4 to 4:1 copolymers of maleic anhydride or acid with another polymerizable ethylenically unsaturated monomer, preferably methyl vinyl ether (methoxyethylene) having a molecular weight (M.W.) of about 30,000 to about 1,000,000. These copolymers are available for example as Gantrez (AN 139 (M.W. 500,000), A.N. 119 (M.W. 250,000) and preferably S-97 Pharmaceutical Grade (M.W. 70,000), of GAF Corporation.

Surfactants (Sudsing Agents)

The present composition optionally comprises a safe and effective amount of a surfactant, in another embodiment comprises from about 0.001% to about 20%, in another embodiment from about 0.05% to about 6%, and in even another embodiment from about 0.1% to about 3% by weight of the composition of surfactant.

Suitable surfactants are those which are reasonably stable and foam throughout a wide pH range and include nonionic, anionic, amphoteric, cationic, zwitterionic, synthetic detergents, and mixtures thereof. Many suitable nonionic and amphoteric surfactants are disclosed by U.S. Pat. Nos. 3,988,433 to Benedict; U.S. Pat. No. 4,051,234, issued Sep. 27, 1977, and many suitable nonionic surfactants are disclosed by Agricola et al., U.S. Pat. No. 3,959,458, issued May 25, 1976, both incorporated herein in their entirety by reference.

Flavoring Agent

The compositions of the present invention may also comprise a safe and effective amount of a flavoring agent. Suitable flavoring agents include oil of wintergreen, oil of peppermint, oil of spearmint, clove bud oil, menthol, anethole, methyl salicylate, eucalyptol, 1-menthyl acetate, sage, eugenol, parsley oil, oxanone, alpha-irisone, marjoram, lemon, orange, propenyl guaethol, cinnamon, vanillin, thymol, linalool, cinnamaldehyde glycerol acetal known as CGA, and mixtures thereof. Flavoring agents are generally used in the compositions at levels of from about 0.001% to about 20%, in another embodiment from about 0.01% to about 5%, in yet another embodiment from about 1% to about 2%, by weight of the composition

Sweetening Agents, Coolants, Salivating Agents, Warming Agents

The present compositions may optionally comprise sweetening agents including sucralose, sucrose, glucose, saccharin, dextrose, levulose, lactose, mannitol, sorbitol, fructose, maltose, xylitol, saccharin salts, thaumatin, aspartame, D-tryptophan, dihydrochalcones, acesulfame and cyclamate salts, especially sodium cyclamate and sodium saccharin, and mixtures thereof. The compositions generally contain from about 0.1% to about 10% of these agents, in another embodiment from about 0.1% to about 1%, by weight of the composition.

In addition to flavoring and sweetening agents, coolants, salivating agents, warming agents, and numbing agents can be used as optional ingredients in compositions of the present invention. These agents are present in the compositions at a level of from about 0.001% to about 10%, in another embodiment from about 0.1% to about 1%, by weight of the composition.

The coolant can be any of a wide variety of materials. Included among such materials are carboxamides, menthol, ketals, diols, and mixtures thereof. Preferred coolants in the present compositions are the paramenthan carboxyamide agents such as N-ethyl-p-menthan-3-carboxamide, known commercially as “WS-3”, N,2,3-trimethyl-2-isopropylbutanamide, known as “WS-23,” and mixtures thereof. Additional preferred coolants are selected from the group consisting of menthol, 3-1-menthoxypropane-1,2-diol known as TK-10 manufactured by Takasago, menthone glycerol acetal known as MGA manufactured by Haarmann and Reimer, and menthyl lactate known as Frescolat® manufactured by Haarmann and Reimer. The terms menthol and menthyl as used herein include dextro- and levorotatory isomers of these compounds and racemic mixtures thereof. TK-10 is described in U.S. Pat. No. 4,459,425, Amano et al., issued Jul. 10, 1984. WS-3 and other agents are described in U.S. Pat. No. 4,136,163, Watson, et al., issued Jan. 23, 1979; the disclosure of both are herein incorporated by reference in their entirety.

Preferred salivating agents of the present invention include Jambu® manufactured by Takasago. Preferred warming agents include capsicum and nicotinate esters, such as benzyl nicotinate. Preferred numbing agents include benzocaine, lidocaine, clove bud oil, and ethanol.

### Composition Use

The frequency of use by the subject is preferably from about once per week to about four times per day, in another embodiment from about thrice per week to about three times per day, in even another embodiment from about once per day to about twice per day. The period of such treatment typically ranges from about one day to a lifetime. The compositions of this invention are useful for both human and other lower animals (e.g. pets, zoo, or domestic animals).

### EXAMPLES

The following non-limiting examples further describe preferred embodiments within the scope of the present invention. Many variations of these examples are possible without departing from the scope of the invention.

The following compositions, made by conventional processing techniques, are described below:

| | Example | | Example | | Example | |
|---|---|---|---|---|---|---|
| | 1 | Example | 2 | Example | 3 | Example |
| | Paste | 1 | Paste | 2 | Paste | 3 |
| | (Second | Gel | (Second | Gel | (Second | Gel |
| | Phase) | Phase | Phase) | Phase | Phase) | Phase |
| Ingredients | % w/w | % w/w | % w/w | % w/w | % w/w | % w/w |
| Sorbitol | 45.402 | 45.502 | 45.402 | 45.502 | 47.902 | 49.202 |
| Precipitated Silica | 22.500 | 22.500 | 22.500 | 22.500 | 20.000 | 20.000 |
| Glycerin | 9.000 | 9.000 | 9.000 | 9.000 | 9.000 | 9.000 |
| USP Purified Water | 8.000 | 8.000 | 8.000 | 8.000 | 8.000 | 8.000 |
| Sodium Lauryl Sulfate | 4.000 | 4.000 | 4.000 | 4.000 | 4.000 | 4.000 |
| Solution | | | | | | |
| Tetrasodium | 3.85 | 3.85 | 3.85 | 3.85 | 3.85 | 3.85 |
| Pyrophosphate | | | | | | |
| Polyethylene Glycol 300 | 3.000 | 3.000 | 3.000 | 3.000 | 3.000 | 3.000 |
| Sodium Acid | 1.000 | 1.000 | 1.000 | 1.000 | 1.000 | 1.000 |
| Pyrophosphate | | | | | | |
| Flavor | 1.000 | 1.000 | 1.000 | 1.000 | 1.000 | 1.000 |
| Carbopol 956 | 0.300 | 0.300 | 0.300 | 0.300 | 0.300 | 0.300 |
| Xanthan Gum | 0.475 | 0.475 | 0.475 | 0.475 | 0.475 | 0.475 |
| Sodium Saccharin | 0.400 | 0.400 | 0.400 | 0.400 | 0.400 | 0.400 |
| Sucralose | 0.030 | 0.030 | 0.030 | 0.030 | 0.030 | 0.030 |
| Na Fluoride | 0.243 | 0.243 | 0.243 | 0.243 | 0.243 | 0.243 |
| Titanium Dioxide | 0.500 | 0.000 | 0.500 | 0.000 | 0.500 | 0.000 |
| Polyethylene¹ | 0.300 | 0.700 | 0.000 | 0.000 | 0.000 | 0.000 |
| Specks/Prills | | | | | | |
| Carnauba Wax¹ | 0.000 | 0.000 | 0.300 | 0.700 | 0.300 | 0.500 |
| specks/prills | | | | | | |
| ¹Carnauba was entrained in FD&C Red No. 40 aluminum Lake (CAS 25956-17-6). | | | | | | |
| | Example | | | Example | | |
| | 4 | Example | Example | 5 | Example | Example |
| | Paste | 4 | 4 | Paste | 5 | 5 |
| | (Second | Gel | Gel | (Second | Gel | Gel |
| | Phase) | Phase | Phase | Phase) | Phase | Phase |
| Ingredients | % w/w | % w/w | % w/w | % w/w | % w/w | % w/w |
| Sorbitol | 45.402 | 46.502 | 46.302 | 47.902 | 49.202 | 49.082 |
| Precipitated Silica | 22.500 | 22.500 | 22.500 | 20.000 | 20.000 | 20.000 |
| Glycerin | 9.000 | 9.000 | 9.000 | 9.000 | 9.000 | 9.000 |
| USP Purified | 8.000 | 8.000 | 8.000 | 8.000 | 8.000 | 8.000 |
| Water | | | | | | |
| Sodium Lauryl | 4.000 | 4.000 | 4.000 | 4.000 | 4.000 | 4.000 |
| Sulfate Solution | | | | | | |
| Tetrasodium | 3.85 | 3.85 | 3.85 | 3.85 | 3.85 | 3.85 |
| Pyrophosphate | | | | | | |
| Polyethylene Glycol 300 | 3.000 | 3.000 | 3.000 | 3.000 | 3.000 | 3.000 |
| Sodium Acid | 1.000 | 1.000 | 1.000 | 1.000 | 1.000 | 1.000 |
| Pyrophosphate | | | | | | |
| Flavor | 1.000 | 1.000 | 1.000 | 1.000 | 1.000 | 1.000 |
| Carbopol 956 | 0.300 | 0.300 | 0.300 | 0.300 | 0.300 | 0.300 |
| Xanthan Gum | 0.475 | 0.475 | 0.475 | 0.475 | 0.475 | 0.475 |
| Sodium Saccharin | 0.400 | 0.400 | 0.400 | 0.400 | 0.400 | 0.400 |
| Sucralose | 0.030 | 0.030 | 0.030 | 0.030 | 0.030 | 0.030 |
| Na fluoride | 0.243 | 0.243 | 0.243 | 0.243 | 0.243 | 0.243 |
| Titanium Dioxide | 0.500 | 0.000 | 0.000 | 0.500 | 0.000 | 0.000 |
| Color | 0.000 | 0.000 | 0.200 | 0.000 | 0.000 | 0.200 |
| Polyethylene ¹ | 0.300 | 0.500 | 0.500 | 0.000 | 0.000 | 0.000 |
| Specks/Prills | | | | | | |
| Carnauba Wax ² | 0.000 | 0.000 | 0.000 | 0.700 | 0.300 | 0.500 |
| specks/prills | | | | | | |

¹ High density polyethylene (CAS 26221-73-8) or low density polyethylene and Yellow No. 10 Aluminum Lake (CAS 688-14-04-0).
² Carnauba wax entrained in FD&C Red. No. 30.

The gel phase and second phase are each separately prepared from the above ingredients, and are charged into an open toothpaste tube via the deep striping method described above. For all examples each phase is used in equal volumes.

While particular embodiments of the present invention have been described, it will be obvious to those skilled in the art that various changes and modifications of the present invention can be made without departing from the spirit and scope of the invention. It is intended to cover, in the appended claims, all such modifications that are within the scope of this invention.

What is claimed is:

1. A multicolored, striped, dentifrice composition comprising:
a. a plurality of stripe phases comprising:
1. at least one first phase that is a translucent or transparent gel phase comprising an effective amount of colorant particles to create a striped appearance in the translucent or transparent gel phase; and
2. at least one second phase;
wherein the first phase and the second phase have contrasting colors and
wherein all of the phases are Theologically equivalent; and
b. a dentifrice dispensing means wherein the phases are in interfacial surface contact with the adjacent phase prior to extrusion from the dispensing means.

2. The composition of claim 1 wherein the colorant particle level is from about 0.1% to about 3% by weight of the composition.

3. The composition of claim 2 wherein the colorant particle level is from about 0.15% to about 2% by weight of the composition.

4. The composition of claim 3 wherein the colorant particle level is from about 0.5% to about 1.5% by weight of the composition.

5. The composition of claim 2 wherein the composition has at least 3 stripe phases.

6. The composition of claim 5 wherein the composition has at least 4 stripe phases.

7. The composition of claim 1 wherein the colorant particle comprises a dye, pigment or lake entrained in a material selected from the group consisting of low density polyethylene (LDPE), high density polyethylene (HDPE), natural waxes, gelling agents, synthetic waxes, water soluble thermoplastic polymers, insoluble binders, water soluble binders, and mixtures thereof.

8. The composition of claim 7 wherein the material is selected from the group consisting of synthetic paraffin wax, synthetic wax, carnauba wax, candelilla wax, purified montan wax, castor wax, paraffin, ceresin wax and bayberry wax, and mixtures thereof.

9. The composition of claim 1 wherein the color of the translucent or transparent gel phase is provided by only the colorant particle.
